(19) United States
(12) Patent Application Publication
(10) Pub. No.: US 2008/0184466 A1
(43) Pub. Date: Aug. 7, 2008

(54) Swimming and dive mask

(75) Inventor: Chih-Cheng Shiue, Escondido, CA (US)

Correspondence Address: LEONARD TACHNER, A PROFESSIONAL LAW;CORPORATION, 17961 SKY PARK CIRCLE, SUITE 38-E, IRVINE, CA, 92614, US

(73) Assignee: QDS Injection Molding, L.L.C.

(21) Appl. No.: 11/701,818

(22) Filed: Feb. 2, 2007

(51) Int. Cl. A61F 9/02

(52) U.S. Cl. 2/428; 2/435; 2/426

(57) ABSTRACT

A water introduction mechanism (either a slit or hole) on the skirt, frame or lens of the mask, which can be opened and closed while the mask is worn to introduce water to wash out fog on the lens. The incoming water may be directed towards the lens by a tab. The water introduction mechanism can be at a variety of different locations on the mask. Where the water introduction mechanism is located on a soft part of mask, (such as the skirt), under normal circumstances, ambient water pressure will prevent the hole from opening. However, under additional external force from the user, the soft part will deform and allow water to enter the mask. The incoming water washes away the fog vapor. The user then ceases applying external force to the soft part, and the hole closes. The water introduction mechanism on the soft part of the mask can also be in the form of a “zipper” or “faucet” type device to selectively admit water into the mask interior while it is one the user's face.

### BACKGROUND OF THE INVENTION

1. Field of the Invention

The present invention relates to a dive or swim mask having a slit or hole located on the mask that can open and close, allowing water to enter the mask when activated by the user. While using the mask during swimming or diving, the mask lens can begin fogging. The user can then activate the slit or hole to allow water to enter the mask, thereby washing out fog vapor. The user then purges the water out of mask.

2. Background of Discussion

When using swimming masks during diving or swimming, the lens of the mask can become fogged. Lens fogging can be caused by a difference in temperature between the inner cavity of the swimming mask and the outside water temperature, or by the user accidentally breathing through his or her nose during use. This fog will block the user's vision, making it impossible to see.

The standard method of preventing fog on the lens is to apply anti-fog solution to the lens before use. However, this is only a temporary solution, and prevents fog from forming only for a short period of time. Additionally, while applying the anti-fog solution, the lens may become scratched, or dust may settle onto the anti-fog coating while the user dries the anti-fog solution.

After the standard swimming mask has already become fogged during use, more experienced users would rapidly separate the mask from their face, allowing water to enter. The user would then rapidly attach the swimming mask back onto their face, preventing additional water from entering the mask. The traditional method of allowing water to enter the mask requires very quick movement, and is difficult to do without flooding the entire inner cavity of the mask with water. The traditional method would also allow water to get into the user's eyes, causing great discomfort. The present invention overcomes these problems through the introduction of water entry slits and tabs which direct the introduced water away from the user's eyes and towards the lens of the mask.

### SUMMARY OF THE INVENTION

The present invention solves the fog on mask lens problem. The invention involves the use of a water introduction mechanism (either a slit or hole) on the skirt, frame or lens of the mask, which can be opened and closed to introduce water to wash out the fog. The incoming water is directed towards the lens by a tab.

The water introduction mechanism can be at a variety of different locations on the swimming mask. The mechanism can be located on the skirt and/or the frame and/or the lens.

Where the water introduction mechanism is located on a soft part of mask, (such as the skirt), under normal circumstances, ambient water pressure will prevent the hole from opening. However, under additional external force from the user, the soft part will deform and allow water to enter the mask. The incoming water washes away the fog vapor. The user then ceases applying external force to the soft part, and the hole closes. The water introduction mechanism on the soft part of the mask can also be in the form of a “zipper” or “faucet”.

Where the water introduction mechanism is located on the lens, the user can activate the mechanism by applying external force, thereby letting water enter the mask. When the user ceases applying external force, the mechanism closes, preventing water from entering the mask.

Where the water introduction mechanism is located on the frame, the mechanism can have a variety of different configurations to allow water to enter mask. These configurations include a type of “zipper” or “faucet.”

The water introduction mechanism can also be located between the lens and the frame or between the frame and the skirt.

The water introduction mechanism is formed so that with external force, the user can activate the mechanism, thereby allowing water to enter the mask.

### BRIEF DESCRIPTION OF THE DRAWINGS

The aforementioned objects and advantages of the present invention, as well as additional objects and advantages thereof, will be more fully understood herein after as a result of a detailed description of a preferred embodiment when taken in conjunction with the following drawings in which:

FIG. 1 is a front view of a user's head having a swimming mask and showing a conventional way of first allowing water in the mask interior to wash away lens fog;

FIG. 2 is a side view of the head and mask of FIG. 1 showing the prior art technique for allowing water in and then expelling it out;

FIG. 3 is a side view of a mask having a nosepiece one-way valve for expelling water from inside the mask;

FIG. 4 is a bottom view of the nosepiece valve of FIG. 3;

FIG. 5 is a side view of the valve of FIGS. 3 and 4 showing the water being expelled at the valve;

FIG. 6 illustrates a first embodiment of the invention shown in a top view thereof;

FIG. 7 shows the activation of the first embodiment using one finger at each slit;

FIG. 8 is a front view of the first embodiment;

FIG. 9 is a front view during activation of the first embodiment;

FIG. 10 is a front view of the first embodiment during exhalation and expulsion through a nosepiece valve;

FIGS. 11 through 16 show various alternative locations for several embodiments of the invention;

FIGS. 17 through 19 show various slit geometries of the invention;

FIGS. 20 through 27 show still other more elaborate slit geometries of the invention; and

FIGS. 28 through 30 show top, side and bottoms views of a zipper-like slider for opening water entry slits of FIG. 26.

### DETAILED DESCRIPTION OF PREFERRED EMBODIMENTS

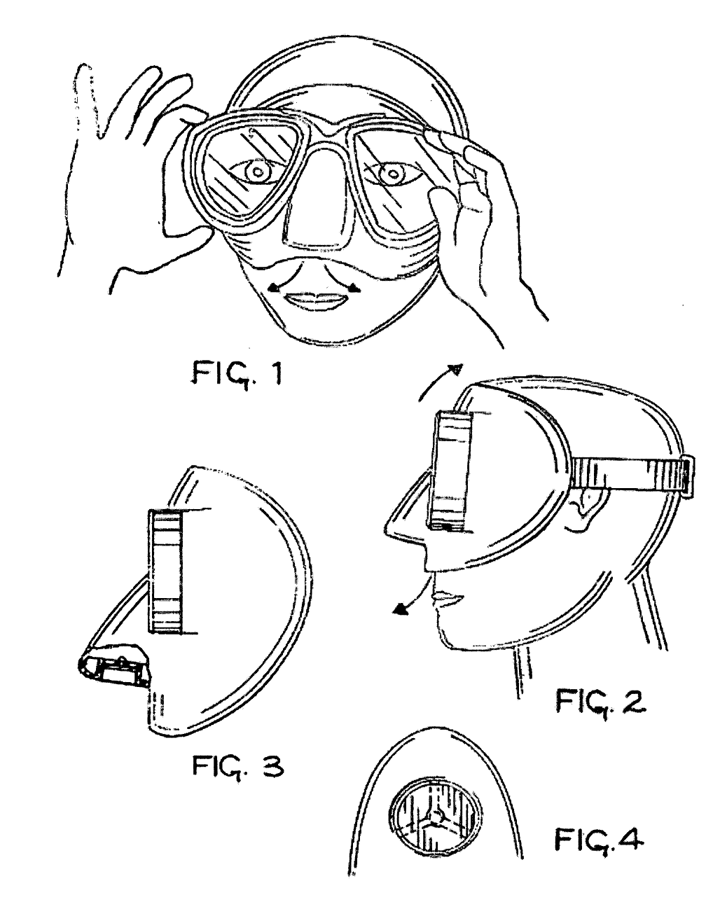

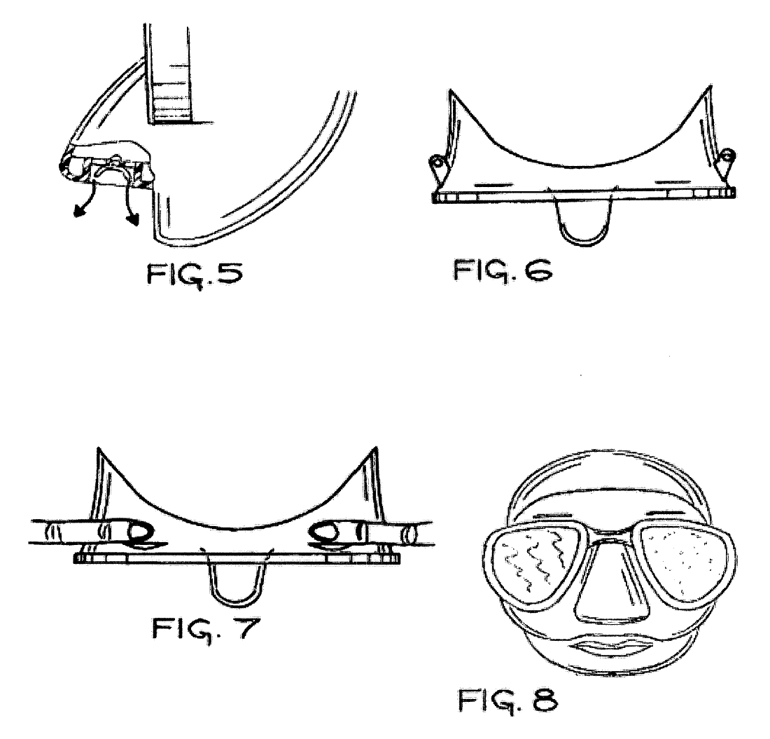

Referring first to prior art FIGS. 1 to 5, it will be seen that a conventional way of defogging the interior surfaces of mask lenses is to rotate the mask on the user's face underwater in the manner shown schematically in FIGS. 1 and 2. This permits a partial flooding of the mask to wash away the fogging vapor. The water is then effectively blown out either through the skirt/user interface or though a nosepiece valve shown in FIGS. 3, 4 and 5. The present invention offers an alternative to this technique using one or more slits or other forms of apertures or openings which allow more controlled water entry under better regulation.

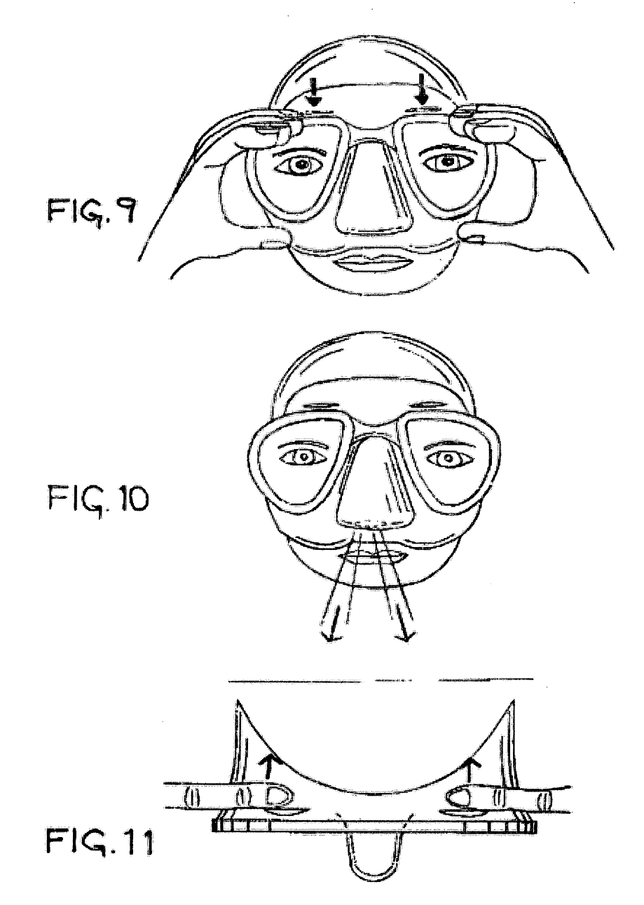

More specifically, referring to FIGS. 6 to 10, it will be seen that a pair of slits is provided in the skirt near the frame above the lenses, one adjacent each lens. These slits are normally kept closed by the pressure of the mask against the user's face caused by the mask strap around the back of the user's head and by the pressure of the surrounding water outside the mask. However, when a user's mask lenses become fogged up (as shown in FIG. 8), the user may open the slits by merely pressing the skirt adjacent the slits as shown in FIG. 7. As a result, water enters the mask interior as depicted in FIG. 9 thereby washing away the fogging vapor. The user may then exhale suddenly or in a pulse-like action to cause the water to exit such as through a nosepiece valve as depicted in FIG. 10. As shown in FIG. 11, the motion of slit or other opening may also be a pulling of the skirt away from the frame using one finger or, as shown in FIG. 12, the slits may be opened by a mutual pulling action between two fingers such as between thumb and forefinger on opposing areas adjacent the slit.

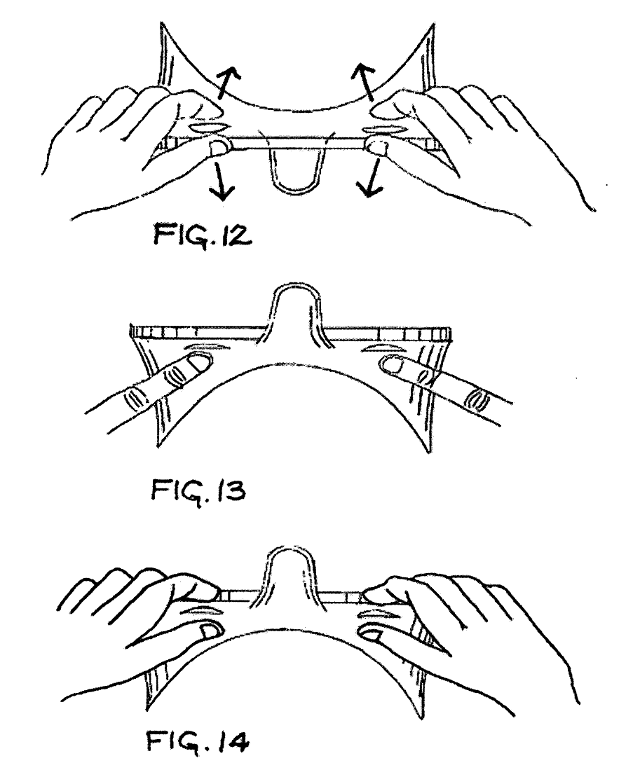

As seen in FIGS. 13 and 14, the slits may also be located on the skirt below the lenses where they may be pressed (FIG. 13) or pulled (FIG. 14) to allow water entry to wash away fogging vapor from the lenses. In addition, as depicted in FIG. 15 and 16, the slits may also be located on the side of the mask on the frame or on the skirt where, again, they may be opened with one finger pressing or two fingers pulling the slits open.

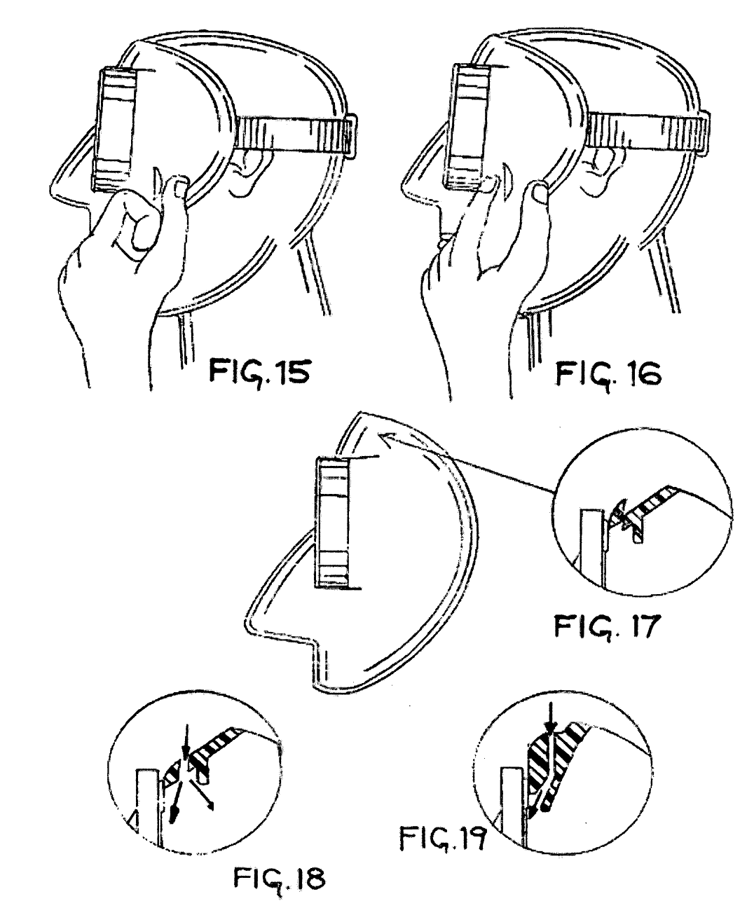

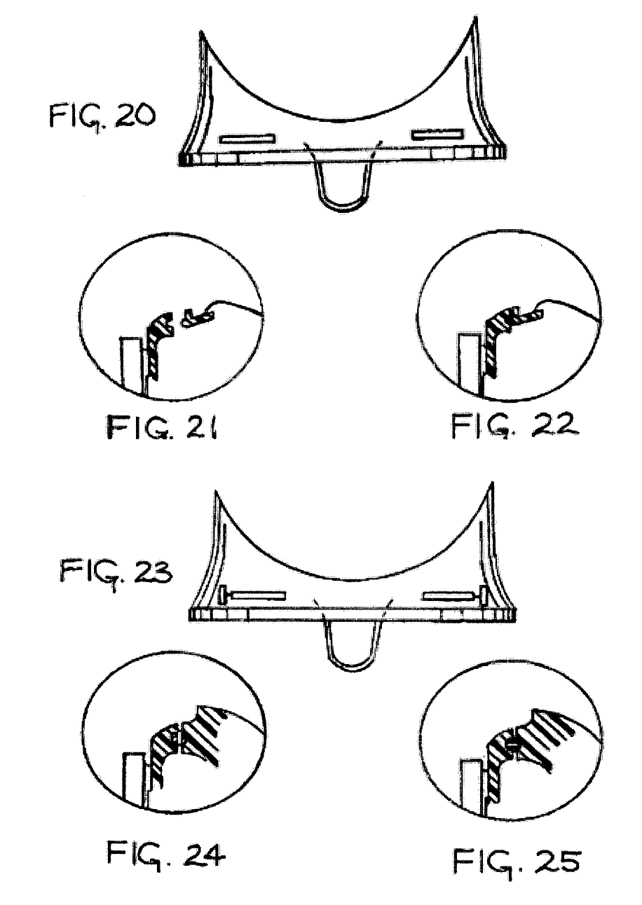

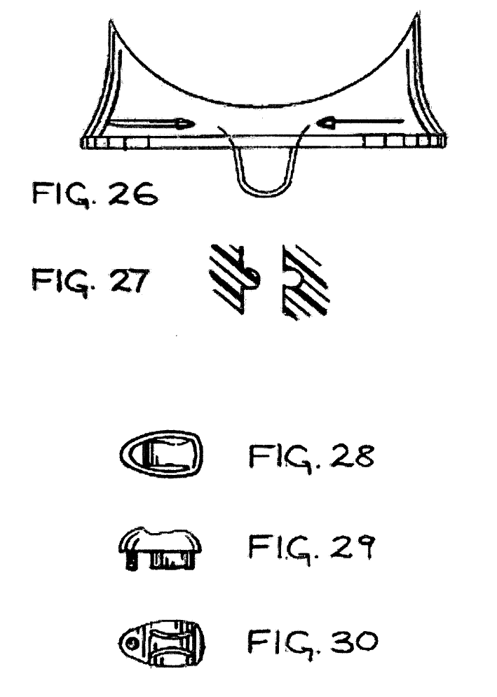

As shown in FIGS. 17 through 30, the slits may be somewhat more sophisticated than mere cuts in the skirt or frame. As shown in FIGS. 17 and 18, there may be a specially shaped bead or a specially shaped channel as shown in FIGS. 19 to direct water flow more toward the lenses. In addition, the slit of FIG. 20 may be of the press-type shown in FIGS. 21 and 22, or of the faucet type shown in FIGS. 23 to 25 or of the zipper-style shown in FIGS. 26 to 30.

More specifically, referring to the press-type slits of FIG. 20, shown in open cross-section in FIG. 21 and closed cross-section in FIG. 22, it will be seen that mere normal force will separate the slit components. On the other hand, the faucet-type slits of FIG. 23 to 25, use a rotatable valve which can be either aligned with the slit to admit water (FIG. 24) or perpendicular to the slit to block water entry (FIG. 25). The zipper-type slits of FIG. 26 employ a zip-like operation much like zip-lock plastic bags as shown in cross-section in FIG. 27. Opening and closing of the zipper-type slits of FIG. 26 is preferably facilitated by a slide device shown in top view, side view and bottom view respectively in FIGS. 28 to 30.

The openings or apertures may also take the form of plunger valves or other various mechanisms for allowing ambient water to enter the mask interior to impinge on the lens or lenses and wash away the fog. The water is then expelled either through a nosepiece valve or by lifting the bottom of the mask during exhalation.

Having thus disclosed various illustrative embodiments, it will now be apparent to those having skill in the relevant arts that various modifications may be made to the invention without deviating from the inventive features thereof. Thus, the scope hereof is limited only by the appended claims.

I claim:

1. A swimming mask having a frame supporting at least one transparent lens and a flexible skirt for sealing engagement with a swimmer's face; the mask comprising:
at least one openable slit in the skirt for selective entry of water into the mask interior for washing away fog on the at least one lens.

2. The swimming mask recited in claim 1 wherein said slit is openable by pressing an area adjacent said slit.

3. The swimming mask recited in claim 1 wherein said slit is openable by pulling apart opposed areas adjacent said slit.

4. The swimming mask recited in claim 1 wherein said slit is openable by a faucet-like control device.

5. The swimming mask recited in claim 1 wherein said slit is openable by a zipper-like control device.

6. The swimming mask recited in claim 1 said slit terminating in a tab for directing entering water toward said lens.

7. A dive mask having a frame supporting at least one transparent lens and a flexible skirt for sealing engagement with a diver's face; the mask comprising:
at least one openable slit in the skirt for selective entry of water into the mask interior for washing away fog on the at least one lens.

8. The dive mask recited in claim 1 wherein said slit is openable by pressing an area adjacent said slit.

9. The dive mask recited in claim 1 wherein said slit is openable by pulling apart opposed areas adjacent said slit.

10. The dive mask recited in claim 1 wherein said slit is openable by a faucet-like control device.

11. The dive mask recited in claim 1 wherein said slit is openable by a zipper-like control device.

12. The dive mask recited in claim 1 wherein slit terminates in a tab shaped to direct entering water toward said lens.

13. A swimming mask having a frame supporting at least one transparent lens and a flexible skirt for sealing engagement with a swimmer's face; the mask comprising:
at least one openable aperture in the frame for selective entry of water into the mask interior for washing away fog on the at least one lens.

14. The swimming mask recited in claim 13 wherein said aperture is openable by pressing an area adjacent said aperture.

15. The swimming mask recited in claim 13 wherein said aperture is openable by pulling apart opposed areas adjacent said aperture.

16. The swimming mask recited in claim 13 wherein said aperture is openable by a faucet-like control device.

17. The swimming mask recited in claim 13 wherein said aperture is openable by a zipper-like control device.

18. A dive mask having a frame supporting at least one transparent lens and a flexible skirt for sealing engagement with a diver's face; the mask comprising:
at least one openable aperture in the frame for selective entry of water into the mask interior for washing away fog on the at least one lens.

19. The dive mask recited in claim 18 wherein said aperture is openable by pressing an area adjacent said aperture.

20. The dive mask recited in claim 18 wherein said aperture is openable by pulling apart opposed areas adjacent said aperture.

21. The dive mask recited in claim 18 wherein said aperture is openable by a faucet-like control device.

22. The dive mask recited in claim 18 wherein said aperture is openable by a zipper-like control device.

23. A swimming mask having a frame supporting at least one transparent lens and a flexible skirt for sealing engagement with a swimmer's face; the mask comprising:
at least one openable aperture in the lens for selective entry of water into the mask interior for washing away fog on the at least one lens.

24. The swimming mask recited in claim 23 wherein said aperture is openable by pressing an area adjacent said aperture.

25. The swimming mask recited in claim 23 wherein said aperture is openable by pulling apart opposed areas adjacent said aperture.

26. The swimming mask recited in claim 23 wherein said aperture is openable by a faucet-like control device.

27. The swimming mask recited in claim 23 wherein said aperture is openable by a zipper-like control device.

28. A dive mask having a frame supporting at least one transparent lens and a flexible skirt for sealing engagement with a swimmer's face; the mask comprising:
at least one openable aperture in the lens for selective entry of water into the mask interior for washing away fog on the at least one lens.

29. The dive mask recited in claim 28 wherein said aperture is openable by pressing an area adjacent said aperture.

30. The dive mask recited in claim 28 wherein said aperture is openable by pulling apart opposed areas adjacent said aperture.

31. The dive mask recited in claim 28 wherein said aperture is openable by a faucet-like control device.

32. The dive mask recited in claim 28 wherein said aperture is openable by a zipper-like control device.

33. An underwater mask for preventing water from coming in contact with the eyes of swimmers, divers, snorkelers and the like; the mask comprising:
a frame supporting at least one transparent lens and a flexible skirt for sealing engagement with a user's face; and
a selectively actuatable device for permitting controlled entry of ambient water into said mask without breaking said sealing engagement, said device providing said controlled entry of water for rinsing said at least one lens when said lens is obscured by fog.
